(19) United States
(12) Patent Application Publication
(10) Pub. No.: US 2023/0190185 A1
(43) Pub. Date: Jun. 22, 2023

(54) HIGH DENSITY EEG SYSTEM FOR PRECISION PSYCHIATRY

(71) Applicant: New York University, New York, NY (US)

(72) Inventors: Dino DVORAK, Brooklyn, NY (US); Andre A. FENTON, New York, NY (US)

(21) Appl. No.: 18/000,212

(22) PCT Filed: Jun. 4, 2021
(86) PCT No.: PCT/US21/35925

§ 371 Date: Nov. 29, 2022

Related U.S. Application Data
(60) Provisional application No. 63/034,943, filed on Jun. 4, 2020.

(51) Int. Cl. A61B 5/00

(52) U.S. Cl. CPC A61B 5/4836; A61B 5/0002

(57) ABSTRACT

A system for precision psychiatry includes a wearable device including a cap and EEG electrodes attached thereto, the cap configured to be placed over a head of a user and including a plurality of first linear actuators configured to expand and contract to increase and decrease a size of the cap, respectively, the EEG electrodes connected to the cap via a plurality of second linear actuators configured to expand and contract to move the EEG electrode toward and away from, respectively, a scalp of the user, a processing device configured to be connected to the EEG electrodes of the wearable device to know the 3-D location of the EEG electrodes and to receive brain activity signals therefrom, the processing device generating a neurofeedback signal based on a detected aberrant brain activity, and a feedback device providing the neurofeedback signal to the user.

### PRIORITY

The present disclosure a National Phase Application of PCT Patent Application Serial No. PCT/US2021/035925 filed Jun. 4, 2021, which claims priority to U.S. Provisional Patent Application Ser. No. 63/034,943 filed on Jun. 4, 2020; the entire disclosure of the above applications/patents is incorporated herein by reference.

### BACKGROUND

One in five people suffers from some form of brain disorder. The most prominent disorders—anxiety and depression are experienced by 42 and 16 million Americans, respectively and are the leading cause of disability and lost productivity, costing annually $42-53 billion. Of those who die by suicide, 90% have an underlying mental illness and suicide is the 10th leading cause of death in the U.S. Although depression and anxiety are the leading cause of disability worldwide, and a major contributor to the global burden of disease, 50% of youth and 60% of U.S. adult sufferers did not receive mental health services in the previous year.

Currently, the first line of intervention for treating brain disorders is pharmacological. However, overprescribed neuropsychiatric drugs such as anxiolytics, antidepressants and Attention Deficit Hyperactivity Disorder (ADHD) medication suffer from inadequate effectiveness, significant side effects, and health problems. Selective serotonin reuptake inhibitors (SSRIs), which are the most commonly prescribed antidepressants, have a side effect incidence of 43% leading to discontinuation of therapy without remission. Benzodiazepines, are the most commonly prescribed anxiolytics and are consumed by 15% of the US population with a 45% incidence of side effects, while leading to addiction at more than 50% of users, by some estimates. Talk therapy, a secondary choice for treating brain disorders, especially anxiety and depression, is also ineffective, requires dozens of visits in the therapist's office and is very expensive. Importantly, both pharmacological and talk therapy are typically prescribed without any quantitative assessment, using trial and error and therefore rely on a subjective evaluation of efficacy. Thus, an increasing number or patients are seeking alternative interventions for brain disorders including depression, anxiety and ADHD.

Precision psychiatry, a subfield of precision medicine is an emerging approach that takes advantage of brain imaging, biomarkers, environmental exposures, genomics, longitudinal data collection, data analysis and large datasets in order to develop novel approaches for treatment and prevention of an individual's psychiatric disorders. However, current precision psychiatry technology is costly and inaccessible to the general public.

### SUMMARY

The present disclosure relates to a system for precision psychiatry, comprising a wearable device including a cap and a plurality of EEG electrodes attached thereto, the cap configured to be placed over a head of a user and including a plurality of first linear actuators, each of the first linear actuators configured to expand and contract to increase and decrease a size of the cap, respectively, the plurality of EEG electrodes connected to the cap via a plurality of second linear actuators, each of the second linear actuators configured to expand and contract to move the EEG electrode toward and away from, respectively, a scalp of the user when the cap is placed over the head of the user while a 3-D location of the plurality of EEG electrodes is known at all times. The system also comprises a processing device configured to be connected to the EEG electrodes of the wearable device to know the 3-D location of the plurality of EEG electrodes and to receive brain activity signals therefrom, the processing device generating a neurofeedback signal based on a detected aberrant brain activity, and a feedback device providing the neurofeedback signal to the user.

The present disclosure is also directed to a high-density EEG wearable device for precision psychiatry, comprising a cap configured to be placed over a head of a user, the cap including a plurality of first linear actuators, each of the first linear actuators configured to expand and contract to increase and decrease a size of the cap, respectively, and a plurality of electrodes attached to the cap, the electrodes connected to one another via the first linear actuators and to the cap via a plurality of second linear actuators, each of the second linear actuators configured to expand and contract to move the electrode toward and away from, respectively, a scalp of the user when the cap is placed over the head of the user.

The present disclosure also relates to a method for autonomously adjusting a high-density EEG wearable device, comprising adjusting a cap of a wearable device to an initial configuration, placing the cap of the wearable device over the user's head, and adjusting the cap of the wearable device so that a plurality of EEG electrodes attached to the cap comfortably contact a scalp of the user, a size of the cap being adjusted via one of an expansion and contraction of first linear actuators connecting the EEG electrodes to one another and a position of the EEG electrodes relative to the scalp of the user being adjusted via one of an expansion and contraction of a second linear actuators connecting the EEG electrodes to the cap.

### BRIEF DESCRIPTION

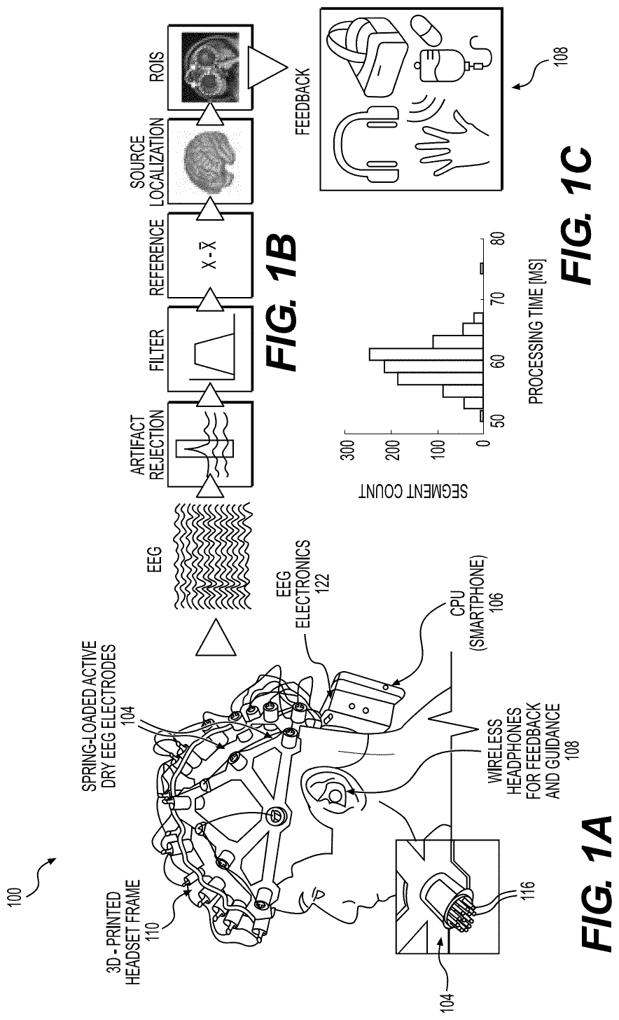

FIGS. 1A-D show a schematic view of a system for precision psychiatry according to an exemplary embodiment of the present disclosure.

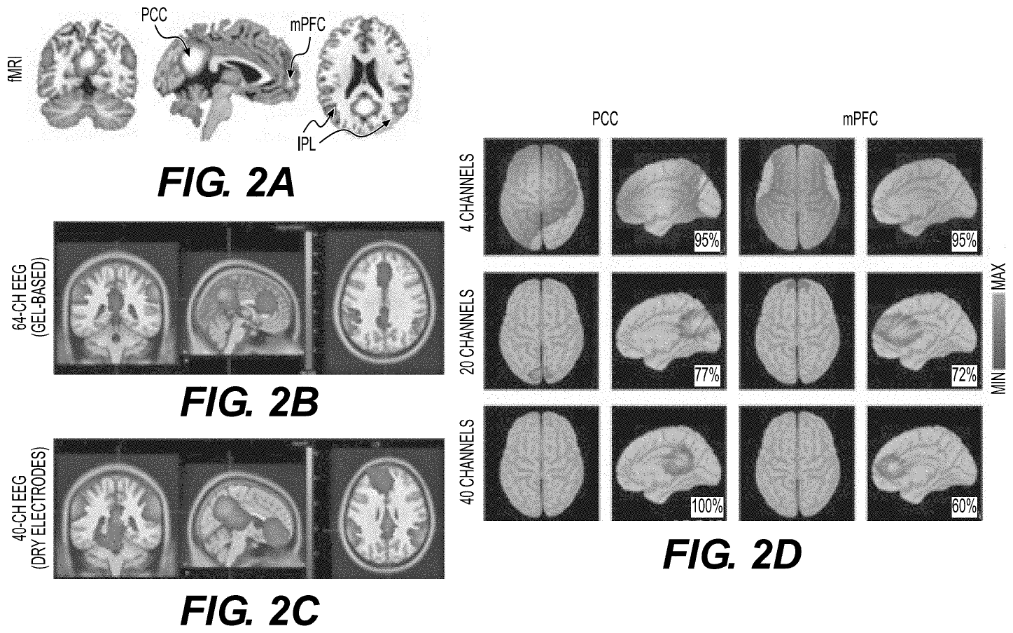

FIGS. 2A-D show image data corresponding to default mode network (DMN) activation findings using A) fMRI, B) a 64-channel gel-based EEG, and C) a 40-channel dry EEG system according to the exemplary embodiment of the present disclosure.

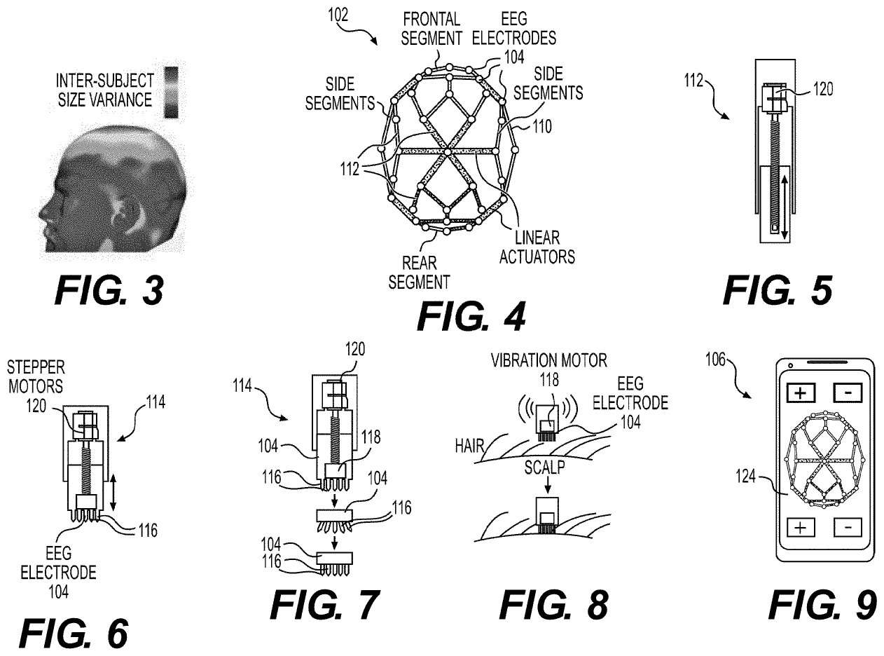

FIG. 3 shows a 3-D head model demonstrating a variance of inter-subject cranium size.

FIG. 4 shows a top plan view of a wearable device according to the exemplary system of FIGS. 1A-D.

FIG. 5 shows a longitudinal side view of a first linear actuator of the wearable device changing wearable size and shape according to the exemplary system of FIGS. 1A-D.

FIG. 6 shows a longitudinal side view of a second linear actuator of the wearable device driving electrodes up and down according to the exemplary system of FIGS. 1A-D.

FIG. 7 shows a longitudinal side view of the third linear actuator of the wearable device moving electrode protrusions according to the exemplary system of FIGS. 1A-D.

FIG. 8 shows a side view of the electrode including a vibration motor according to the exemplary system of FIGS. 1A-D.

FIG. 9 shows a plan view of a smartphone including a graphical user interface according to the exemplary system of FIGS. 1A-D.

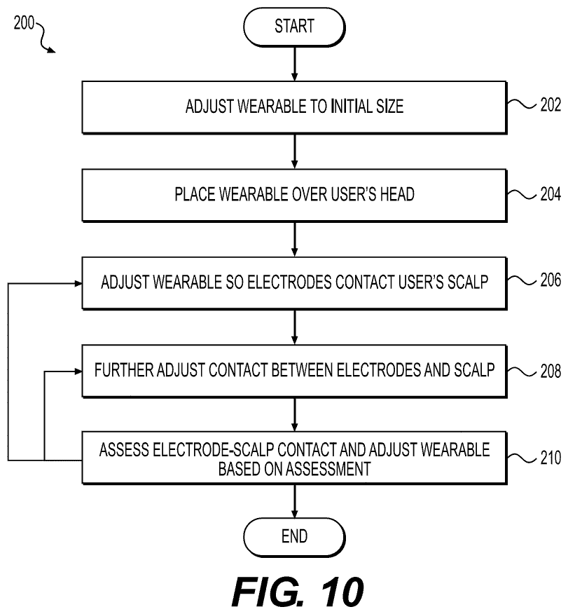

FIG. 10 shows a flow diagram of a method according to an exemplary method of the present disclosure.

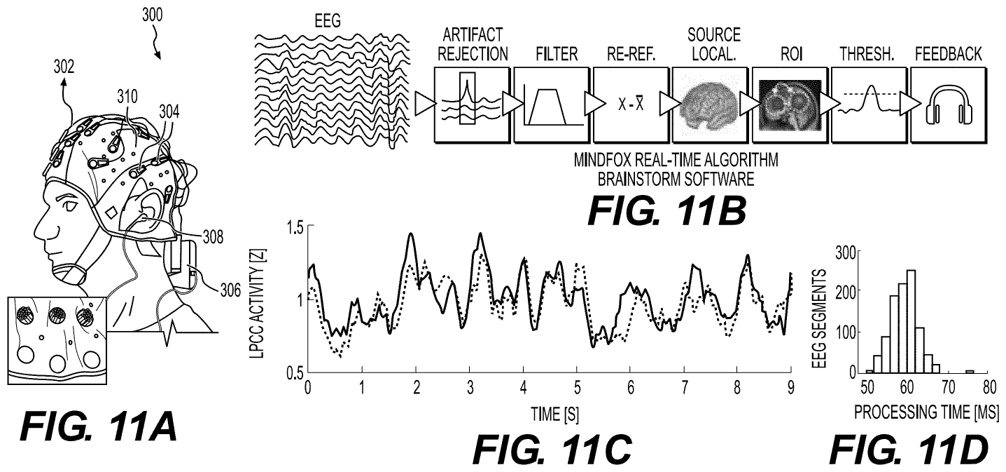

FIGS. 11A-D show a schematic diagram of a system for precision psychiatry according to another exemplary embodiment of the present disclosure.

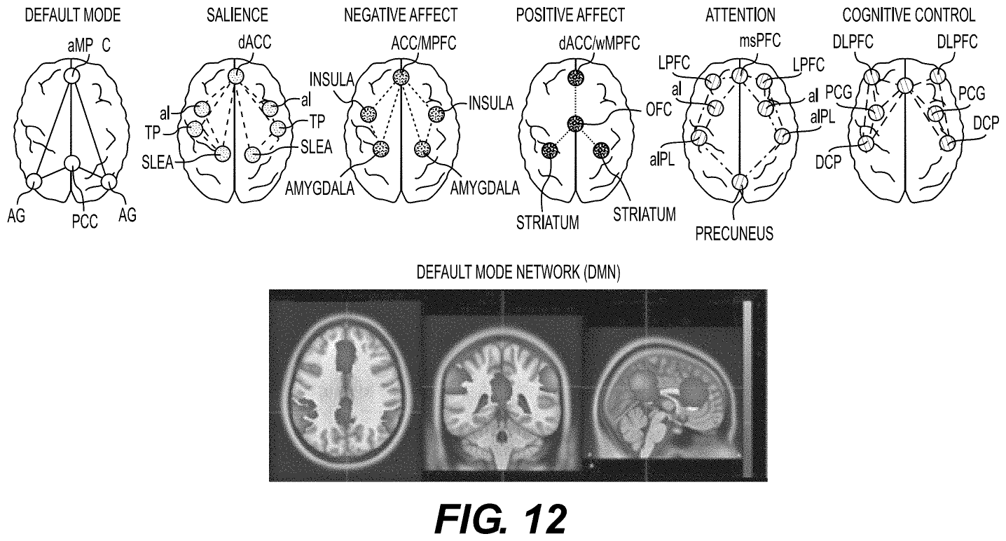

FIG. 12 shows a schematic diagram of spatial activation patterns of functional brain networks.

### DESCRIPTION

The present disclosure may be further understood with reference to the following description and the appended drawings. The present disclosure relates to a system and method for providing precision psychiatry and, in particular, relates to a neurofeedback system and method that measures a user's (e.g., patient's) aberrant brain activity, which is indicative of disorder symptoms, to train the user to volitionally control their brain activity using perceptible stimuli, including audio, visual and/or tactile feedback to alleviate symptoms of brain disorders. Exemplary embodiments of the present disclosure comprise an EEG-based consumer wearable, which self-adjusts so that EEG sensors are in a desired contact with the user's scalp. Thus, the wearable may be used directly by the user without requiring technician setup and intervention. Once the wearable has been adjusted to the user, brain activity detected via the EEG sensors may be transmitted in real-time to a smartphone, tablet, or other portable processing device so that feedback based on the detected brain activity may also be provided to the user in real-time.

The EEG-based consumer wearable of the present disclosure can in principle offer non-invasive and inexpensive ways to measure brain function and can deliver closed-loop interventions including neurofeedback. Because aberrant, unwanted brain activity manifests as the disorder symptoms, measurement of such brain activity can be used to influence the user's experience using perceptible audio, visual or tactile signals. These signals may be used to train the user to volitionally control their experience and with it, their own brain activity, thereby alleviating symptoms of brain disorders.

Over 150 providers and consumers from both mental health and wellness domains were interviewed to identify three classes of consumers that may particularly benefit from the EEG-based consumer wearable—therapists, mental health consumers and health-conscious consumers. Therapists can be considered as early adopters, because some of them already use simple neurofeedback technology. Many clinical psychologists or psychiatrists, typically early in their career, are innovators, eager to offer cutting edge technology in their own practice. They want to differentiate themselves from their peers by providing data-driven and/or nonpharmacological interventions. The EEG wearable has the added benefit of allowing the therapists to treat more patients at a time, thereby increasing income for these therapists. Mental health consumers conscious of the side effects of existing pharmacological interventions such as antidepressants or anxiolytic drugs and who do not appreciate time-consuming talk therapy may seek alternative interventions that provide longitudinal health data similar to fitness trackers that can be used anywhere. Parents of children suffering from ADHD represent a special subgroup of mental health consumers who are conscious of the serious side effects of existing ADHD medicine and therefore prefer alternative, nonpharmacological interventions. Wellness and fitness studios catering to health-conscious individuals may also seek integrating mental wellbeing into their existing offerings.

There is currently a large gap between the clinical laboratory technology used to understand brain disorders and the consumer-oriented neurotechnology that might be able to provide treatment. The “precision psychiatry” framework conceptualizes and understands brain disorders at the level of an individual patient's functional brain networks. Functional brain networks may identify interacting brain regions corresponding to cognition/behaviors. Functional brain networks that mediate salience, attention, cognition and affect are the most promising targets for both assessment and therapy. One such network is the default mode network (DMN), that is currently only measured by fMRI, MEG, or research-grade EEG that are both expensive and impractical for consumer-oriented applications. Excessive activation of DMN has been associated with multiple brain disorders including depression and anxiety.

Real-time fMRI neurofeedback, a type of closed-loop intervention, uses a fMRI scanner to assess activity in specific functional brain networks such as the DMN and translates this activity in real-time to the user in form of auditory and/or visual information that the user can volitionally control and therefore with it control the activation of the particular functional brain network from which the neurofeedback signal is derived, thereby reducing symptoms of brain disorder. This approach has been validated using real-time fMRI in proof-of-concept studies for reducing symptoms of depression, anxiety, post- traumatic stress disorder, and ADHD. However, fMRI is extremely expensive and impractical for both therapist- and consumer-oriented applications.

In contrast, therapists and consumers currently only have access to primitive “conventional neurofeedback” technology that is compromised by design, because it typically uses less than 10 EEG sensors to assess brain state. This limits the technology to spectral biomarkers that are disembodied from the underlying anatomical generators and cannot be effectively isolated from environmental and muscle artifacts with an overlapping spectral footprint. In these “conventional neurofeedback” systems, the user is trained to influence signals that lack functional specificity and which can't be effectively dissociated from interfering environmental and muscle activity. As a consequence, unsurprisingly, triple-blind studies comparing conventional neurofeedback therapy with placebo have not shown or found any significant effects.

As shown in FIGS. 1A-D, a neurofeedback system 100 according to an exemplary embodiment comprises a hdEEG (high-density electroencephalography) wearable 102 that meets the unmet need and closes the precision psychiatry gap. In particular, the system 100 acknowledges the needs of therapists, mental health consumers and wellness consumers directly to provide an inexpensive wearable system for real-time functional brain imaging comparable to fMRI and closed-loop interventions for diagnosis, treatment, and assessment of depression, anxiety and other brain disorders, including mild traumatic brain injury. The wearable 102 includes a plurality of electroencephalogram (EG) electrodes 104 connected to a processing device 106. The processing device 106 receives the EEG signals and generates feedback signals to be delivered to the user using sound, a virtual reality experience, tactile stimulation via, for example, headphones, a VR headset, tactile transducers or other feedback devices and/or pharmacological intervention, for example intra-venous (IV) drips, pill and liquid delivery systems or inhalers for closed-loop drug delivery using, e.g., a feedback device 108.

The wearable 102 closes the precision psychiatry gap via several key features. First, the wearable 102 uses an order of magnitude of more EEG electrodes 104 than existing consumer-oriented neurotechnology. This enables a quantum leap from disembodied 1-D spectral estimates derived from EEG signals to 4-D imaging of brain activity in the cerebral volume and across time, functionally similar to fMRI, but with superior temporal resolution at a millisecond timescale, therefore enabling real-time assessment of functional brain networks such as DMN that are directly impacted in brain disorders such as depression and anxiety. Furthermore, precise localization of underlying brain generators allows elimination of muscle artifacts localized outside the brain.

Second, while research-grade hdEEG systems with comparable imaging capability of the presently disclosed wearable require multiple components such as cap and external amplifiers that are typically interconnected by several dozens of wires, a desktop computer with specialized software to operate, the wearable 102 may utilize an inexpensive, yet high resolution processing device 106 including EEG electronics 122 combined with inexpensive smartphone technology integrated into the compact headset to run real-time source localization algorithms, therefore enabling further price reduction, complexity reduction and deployment directly to consumers.

Third, the processing device 106 may be programmed with an automatic innovative algorithm that can change a shape of the wearable 102 to fit most users, move the electrodes 104 closer to the scalp to improve contact, change shape of EEG protrusions and mechanically vibrate the electrode 104 to better penetrate hair and improve conductive electrode contact as well as guide the user to a particular electrode that needs improving the contact with the scalp, using feedback that is tactile or visual with, for example, a LED, color-changing polymer, or other means. When, for example, one of the electrodes 104 vibrates, it alerts the user that particular electrode 104 is not in a desired contact with the scalp, thereby guiding the user to manually adjust the vibrating EEG electrode 104 that the user might not be able to see directly because of its location. Thus, the wearable 102 can be setup without external help to enable consumer-oriented applications and at-home delivery of intervention. Visual feedback may function similarly to the tactile feedback, alerting the user that certain EEG electrodes are not in a desired contact with the scalp. Visual feedback may also alert, for example, a therapist, aide or other helper that may also be present during use, that a particular EEG electrode requires adjustment relative to the scalp. The wearable 102 of the present disclosure drastically reduces headset cost without sacrificing the number of sensors required for robust 4-D imaging of functional brain networks and for maintaining signal quality while operating in real-time, while designing headsets that can be adjusted without external help either by a fully autonomous setup operation, or semi-autonomously by guiding the user through an adjustment procedure.

Fourth, hdEEG systems capable of source localization of brain activity typically require a precise capture of electrode locations relative to the user's scalp for devising the forward model used in source localization algorithm. Electrode locations are typically acquired using 3D optical digitizers such as Structure Sensor Mark II by Occipital or by using fMRI image-based electrode localization. The wearable 102 solves this limitation by utilizing a rigid frame with embedded spring-loaded electrodes whose precise location relative to the frame can be captured using linear displacement sensors embedded in the electrode housing. In such a way, locations of the EEG electrode relative to the frame and its initial, factory state is known even after placing the wearable 102 over user's head and adjusting displacement of electrodes either using linear actuators 114 or by spring action as electrode lightly presses against the scalp. Furthermore, because the amount of linear displacement of linear actuators 112 for adjusting shape and size of the wearable 102 can be precisely controlled and captured by the processing device 106, the absolute 3-D electrode locations are known via the processing device 106 at all times and therefore can be used for updating forward model used in source localization algorithm. The wearable 102 can also utilize variety of precomputed forward models that can be evaluated during use based on best match to expected patterns of activity.

In contrast to current commercial products, the system 100 is capable of providing real-time access to functional brain network biomarkers such as DMN. Current commercial products are incapable of such real-time assessment largely because of their use of a small number of EEG sensors that don't allow precise source localization of functional brain networks that in the precision psychiatry framework, are dysfunctional in brain disorders such as depression and anxiety. In an exemplary embodiment, the system 100 utilizes the exemplary 40-channel dry EEG electrodes 104, allowing the algorithms in the wearable of the present disclosure to detect DMN in real-time while running on inexpensive hardware which enables both assessment of functional brain network biomarkers and closed-loop interventions such as source-based neurofeedback that has been already validated using fMRI technology. The source localization also effectively separates the brain-derived electrical signals from extrinsic artifact signals that would otherwise reduce specificity of the feedback signal.

The system 100 provides a consumer oriented hdEEG system with fMRI-like 4-D imaging functionality but for a fraction of cost. The ability to substantially reduce the cost of the hdEEG wearable of the present disclosure is enabled by the software operating on ubiquitous and thus, inexpensive processing devices 106 such as, for example, smartphone devices and/or tablet devices that have sufficient computational power for real-time source localization. The system 100 may utilize a B2B subscription-based business model driven primarily by per-use fees rather than sales of EEG systems to therapists which is the case for all conventional neurofeedback systems providers. Therefore, the cost-of-goods (COGS) have to be sufficiently low to enable the subscription model.

The wearable 102 may be setup without external help. Currently, all hdEEG headsets that allow source localization need to be setup by an EEG technician who places the EEG sensors at specific locations in a time-consuming and uncomfortable process that often requires placing conductive gel between the EEG electrodes and scalp, which is unacceptable for most therapist- and consumer-oriented applications. The wearable 102 of the present disclosure, however, can be setup without external help. Specifically, as described above, according to one embodiment, the wearable 102 may utilize the dry electrodes 104, which are applied to the scalp of a patient without the use of an electrolytic gel. Rather, the electrodes 104 are comprised of a metal which acts as a conductor between the electrode and the scalp. In another embodiment, however, the wearable 102 may utilize gelled and/or saline electrodes to facilitate conductivity. The wearable 102 may be adjusted either autonomously by measuring electrode impedance and signal quality and change headset shape, distance between the electrode 104 and the scalp, move electrode protrusions and mechanically vibrate the entire electrode 104 to improve contact with scalp or semi-autonomously, by guiding the user to adjust the specific electrodes 104 using tactile feedback using mechanical vibrations, or visual signaling with a LED, color-changing polymer, or other means of identifying the electrodes 104 that need improving contact with the scalp. Finally, the wearable 102 can improve and maintain contact with the scalp for persistent closed-loop electrode quality maintenance and overall comfort, as described in U.S. Pat. No. 9,408,575 and U.S. Publ. Appln. No. 20120143020A1.

Because all conventional neurofeedback systems require a substantial hardware purchase and relatively long training to use it, becoming a neurofeedback therapist is an obstacle for therapists interested in neurofeedback technology. The system 100 may bypass this obstacle by providing therapists with the hardware on a subscription basis and shares the profit from each use by charging a per-use fee from each training session. This model allows therapists to start with the technology without a substantial initial investment while at the same time allowing to track technology usage and continuously improve the algorithms.

As described above and as shown in FIGS. 1A-D, the system 100 may include, in one exemplary embodiment, a 40-channel battery operated, wearable hdEEG system. In particular, the wearable 102 may include dry EEG electrodes 104 embedded in an inexpensive 3D-printed cap 110 with EEG electronics 122 and connected to an inexpensive processing device 106 (e.g., smartphone). Analog EEG signals are sampled using a low-noise analog front-end and relayed via a wired or wireless interface to the processing device 106. Signals undergo real-time pre-processing including artifact removal, band-pass filtering, re-referencing and are submitted to a real-time source-localization algorithm, which maps the EEG signals to their cerebral sources (FIG. 1B). Thanks to extensive algorithmic optimization, the entire signal processing chain operates in real-time. Each 250 ms block of 40-channel data is processed in 60 ms on average (FIG. 1C). Pilot data using the dry hdEEG system validated the system's capability of extracting a DMN pattern of activity that is comparable to a fMRI system or research grade 64-channel gel-based (wet) hdEEG system (see FIG. 2B). Although the exemplary embodiment specifically shows and describes the system 100 as including 40 EEG electrodes, the system 100 may comprise any number of EEG electrodes so long as the system 100 is configured to provide a high density EEG system. In one embodiment, the system 100 may comprise between approximately 20-60 EEG electrodes. In another, embodiment, the system 100 may comprise between approximately 30-60 electrodes. A number of the electrodes 104 may be varied depending on any of a number of factors including, for example, a head size of a patient (e.g., a child sized head as opposed to an adult sized head) as well as a functional brain network being identified via the hdEEG system.

Real-time assessment of the functional brain network pattern of activity enables closed loop interventions including neurofeedback, where the neurofeedback signal is derived from one or multiple identified regions of interest (ROI) which can be either predefined or established during ˜2 min baseline for each subject. The real-time algorithm converts instantaneous average power in the ROI into a continuous sound (white noise) with varying intensity which is delivered to the user through the feedback device 108 (e.g., headphones) connected to the processing device 106 (e.g., smartphone). The user attempts to volitionally influence the auditory signal and with it also the brain areas from which the neurofeedback signal is derived.

Commercially available neurofeedback systems operate by converting brain activity to an audio or video signal so patients can learn how to influence this signal and therefore their own brain activity. This approach is similar to use of modern neuroprosthetics, but instead of controlling an artificial limb, patients learn how to control a signal that corresponds to their own brain activity. As discussed above, however, conventional neurofeedback systems have proven inadequate for a variety of reasons. In particular, conventional neurofeedback systems operate almost exclusively in so-called “sensor-space.” In this scenario, a neurofeedback signal is derived directly from the electrical activity recorded from single or multiple EEG sensors.

Most inexpensive, consumer-oriented neurofeedback systems (e.g., Muse by Interaxon; Tab. 1) use gamified neurofeedback, where users are rewarded by sounds when they maintain a predefined physiological profile for prolonged periods of time. Neurofeedback systems used by therapists use either proprietary algorithms (e.g., NO3 by NeuroOptimal; Tab. 1), where the principle of deriving the neurofeedback signal is not known or some form of spectral measure to derive the feedback signal (e.g., NeXus-32 by MindMedia; Tab. 1) including amplitude of a selected spectral component (Amplitude Training; AT), amplitude of the slow (<1 Hz) EEG component (Slow Cortical Potentials; SCP), 12-15 Hz EEG component recorded from a single electrode placed above primary motor cortex (Sensimotor Rhythm; SMR), ratio of two spectral components (Theta-Beta Ratio; TBR) or z-scored activity averaged from multiple electrodes (Z-score; Z).

TABLE 1
Neurofeedback systems.

| Company | System | Channels | Sensors | NF type | Price |
|---|---|---|---|---|---|
| Interaxon | Muse | 2 | dry | game | $300 |
| NeuroOptimal | NO3 | 2 | wet | proprietary | $8,000 |
| MindMedia | NeXus-32 | 21 | wet | AT, SCP, Z | $17,000 |

AT = amplitude training, SCP = slow cortical potentials, Z = z-score. Prices don't include software and computer.

Unsurprisingly, low functional specificity of generated feedback signals in conventional neurofeedback systems is reflected in low efficacy of existing neurofeedback therapy and consequently low penetration of this technology among therapists. Currently, less than 0.5% of therapists utilize conventional neurofeedback in their practice and virtually no consumers directly. There are two reasons why conventional neurofeedback has failed, despite trying for 50 years (indicating substantial market interest). First, conventional neurofeedback uses non-specific EEG spectral components as a proxy for brain function and therefore trains the user to arbitrarily increase or decrease the spectral property of the measured signal. However, the EEG signal is a linear mixture of many ongoing processes originating both inside and outside of the brain.

As an example, increased theta (4-7 Hz) amplitudes are regarded by neurologists as pathological, yet their increase has been observed in long-term meditators; such a signal lacks both a clear physiological origin and functional specificity. Second, the small number of EEG sensors typically used in conventional neurofeedback systems (majority of systems use 1-5 sensors; Tab. 1) cannot localize signals to their cerebral origin and cannot effectively separate the brain's intrinsic electrical activity that is the principal target of neurofeedback and extrinsic electrical activity generated by muscle movements. In this scenario, the user is trained by a neurofeedback signal that is a linear mixture of a non-specific brain signal and muscle activity. Those are the likely reasons, why triple-blind studies comparing conventional neurofeedback therapy with placebo did not find any significant differences, why conventional neurofeedback has not been FDA-cleared for treatment of any neuropsychiatric disorder, and why conventional neurofeedback never reached true commercial success.

Advances in brain imaging provide new insights into functionally-defined brain networks that underlie cognitive, emotional and self-reflective functions and point to the dysfunctions within and between those networks as a potential underlying cause of brain disease. It is important to stress that a functional brain network is not merely identification that an anatomically-defined region of the brain is active, rather it is identification of a specific pattern of temporally-defined coactivation of multiple anatomically-defined brain regions. It is the pattern of coactivation that defines something like the default mode network (DMN) as a functionally-defined network of brain regions. Accordingly, in the precision psychiatry framework, functional brain networks that mediate salience and attention, along with cognition- and affect-mediating networks are the most promising targets for both assessment and therapy.

The most studied network, the DMN has been associated with brain disorders including depression, anxiety, addiction disorders, obsessional disorders, bipolar disorder, attention-deficit/hyperactivity disorder, post-traumatic stress disorder, mild traumatic brain injury, schizophrenia, and autism. The DMN comprises activation of posterior cingulate cortex (PCC), medial prefrontal cortex (mPFC), hippocampus and amygdala, regions associated with cognition and affect. Numerous studies found altered functional DMN connectivity in depressed patients, specifically increased functional connectivity in anterior regions of DMN and decreased functional connectivity in posterior regions of DMN. Electroconvulsive therapy, that is proven to have antidepressant effects results in increased theta phase synchronization between the posterior cingulate cortex (PCC) and the anterior frontal cortex, which are both parts of the DMN. Similarly, patients receiving deep brain stimulation to treat depression showed increased glucose metabolism and regional cerebral blood flow in PCC. While there is growing evidence that points to the disbalance of functional brain networks as a potential underlying cause of mental disease, those findings are not effectively translated into actionable clinical tools that would enable precise assessment and targeted therapies.

The majority of knowledge about functional brain networks has been generated using fMRI technology. Furthermore, real-time fMRI neurofeedback, where activity in functional brain networks is translated in real-time to the patient as video or sound so they can learn how to control this activity and therefore their own brain function has been validated in proof-of-concept studies for reducing symptoms of depression, anxiety and other disorders including post-traumatic stress disorder (PTSD), schizophrenia, addiction and ADHD. However, fMRI is extremely expensive and impractical for both therapist- and consumer-oriented applications. On the other hand, high-density (>32 EEG sensors) electroencephalography (hdEEG) combined with source-localization replicates fMRI DMN findings and consequently provides an inexpensive and effective alternative to fMRI with outstanding temporal resolution that is crucial for assessing the temporal dynamics of brain networks that are needed to guide real-time interventions (FIG. 2A).

In particular, FIG. 2C shows that DMN activity can be estimated using the exemplary 40-channel dry EEG electrodes. FIGS. 2A-D show a DMN pattern of activity estimated using A) fMRI (FIG. 2A), B) 64-channel gel-based EEG from a publicly available resting state dataset (FIG. 2B) and C) using the exemplary 40-channel dry EEG system 100 (FIG. 2C). Source-localized EEG activity maximally similar to midline DMN structures—PCC and mPFC (D)—obtained by subsampling 64-ch EEG data to 4-ch EEG (top), 20-ch EEG (middle) and 40-ch hdEEG (bottom) with percentage of subjects (total 22 subjects) with the identified DMN component (FIG. 2D). Only the 40-ch hdEEG identified well-localized DMN midline structures (especially PCC) in 100% of subjects. FIG. 2D illustrates that the ability to detect DMN depends on electrode placement and impedance quality. Noisy electrodes must be removed due to unsatisfactorily high impedance and poorly placed electrodes must also be removed because they violate assumptions of the forward model upon which source localization is based. As illustrated, fewer electrodes make it harder or impossible to detect DMN activity. Accordingly, the ability to detect DMN activity serves as an assay for assessment of the hdEEG signal quality.

Compared to conventional neurofeedback that operates directly in the sensor space, the functional brain network neurofeedback of system 100 operates in source space, which has several key advantages. First, it estimates the spatiotemporal dynamics of neuronal currents generated by synchronous dendritic activity within anatomically localized networks of neurons in the brain, therefore enabling estimation of the activity of functional networks that are defined by inter-regional synchrony, and these networks have been identified to underlie both brain function as well as brain disorders such as depression and anxiety. Feedback from activation of a functionally-defined brain network such the DMN might then be used to assess a functional state of the brain, the brain's inter-regional integrity, or alter the activity of the network itself and consequently improve the neuropsychological state. Second, compared to conventional sensor-space neurofeedback, source-based neurofeedback can effectively separate the brain's intrinsic signals from extrinsic signals because those signals can be localized in space, i.e., proximal to their muscle generators outside of the brain. This results in a functionally more specific feedback signal that is derived from functionally- and anatomically-specific brain regions while excluding interference from generators localized outside the brain. Third, anatomically-localized patterns of functional brain network activity such as DMN that is localized in the cortical midline structures including PCC and mPFC provide independent validation of signal fidelity. If a typical network coactivation pattern cannot be observed, the EEG signal quality can be deemed insufficient to decode a particular functional brain network and therefore whatever feedback signal is generated will necessarily lack the required specificity.

In contrast, in sensor-space conventional neurofeedback, the electrical signal on an EEG electrode can be detected at all times and its functional specificity cannot be independently validated. Fourth, activity within functional brain networks can be used as functional biomarkers for a variety of biotypes including rumination, anhedonia, anxious avoidance, negative bias, threat dysregulation, context insensitivity, inattention and cognitive dyscontrol. As an example, rumination can be defined as increased connectivity (temporal coactivity) between DMN nodes (PCC and mPFC) observed at rest. The pattern of activity and synchronization between nodes of functional brain networks provide a multidimensional biomarker which can be used for disorder classification, monitoring treatment efficacy and guiding recommendations for treatment, as required by precision psychiatry.

The electrode interfaces used in the majority of commercial EEG electrodes are typically “wet”, utilizing water-based conductive gels, which provide coupling between the electrode surface and the scalp. Before EEG signals can be recorded, a conductive gel needs to be applied individually to each electrode in a ˜30 min (64ch EEG system) time-consuming and uncomfortable process. “Dry” EEG electrodes are typically used in consumer EEG devices, but increasingly also in research EEG devices. The dry electrode surface directly interfaces with the scalp. The impedance of dry EEG electrodes typically ranges from hundreds of kOhm to a few MOhm compared to ˜10 kOhm in gel-based electrodes. To maintain a stable connection within a reasonable (<50 kOhm) impedance range, dry EEG electrodes require maintaining contact with the skin. This is typically provided by embedding electrodes in a solid harness which is tightly fitted around the scalp, or by embedding electrodes in a stretchable fabric that presses the electrodes towards the scalp, or by using electrodes with preloaded pins which press against the scalp. Dry EEG sensors are typically made from a flexible polymer bristle coated with Ag/AgCl or alternatively with spring-loaded gold-coated contacts which result in comparable performance. The high electrode impedance of dry EEG sensors makes wire connections between the electrodes and the analog-to-digital converter (ADC) that is highly susceptible to electromagnetic interference, which is typically solved either by active shielding of signal pathways or placing the impedance matching circuit proximal to the electrode to reduce electrode output impedance.

Commercially available dry EEG headsets have several drawbacks that prevent using them in consumer-oriented settings. First, the existing dry EEG headsets are extremely expensive, with prices ranging from $40,000 to $85,000. Second, they require additional purchase of the computer (e.g., $2,000-$5,000) and specialized software for source localization and neurofeedback (e.g., $3,500 Neuroguide by Applied Neuroscience), which makes the headset not only more expensive, but also much more complex to use. Third, they have to be shipped in at least three different sizes in order to accommodate the majority of cranium circumferences. Fourth, they require a second person—the “EEG technician” for their setup in order to assure symmetrical placement on the scalp and good signal quality before starting a recording. Finally, they are primarily used to generate data that are processed offline as opposed to in real-time, as required by neurofeedback applications. This assumes stationarity of the signal during recording, which is rarely the case in real-world settings where the subject can move, speak, clench teeth or even walk during the session.

The system 100, however, overcomes those obstacles by introducing several significant innovations. The ability to substantially reduce the cost of the EEG system is enabled primarily by utilizing 3D-printing technology for manufacturing the cap 110 and integrating the processing and source localization unit within the wearable by utilizing a processing device 106 such as, for example, a smartphone device that is ubiquitous, inexpensive, and can perform computationally-optimized real-time source localization. Second, the present disclosure includes autonomous procedures, which allows both resizing of the headset to fit each subject, as well as autonomous procedures for improving sensor contact with the scalp, therefore eliminating the need of multiple headset sizes to accommodate all head shapes and sizes and optimizing user comfort by adjusting pressure between electrode and scalp. Together, these efforts will enable consumer-oriented applications that don't require assistance by another person.

An EEG headset with imaging capability that can be used directly by consumers must meet several technical requirements. First, the number of sensors used in the headset has to be large enough, to precisely assess functional brain networks including DMN. Second, the headset needs to accommodate most head shapes and sizes, as shown in FIG. 3, and therefore needs to be able to physically adjust while electrode locations relative to the scalp are known at all times via the processing device 106. Third, it has to be possible to setup the headset without the help of another person in a relatively short time to avoid user's frustration. Fourth, the headset has to autonomously self-adjust before as well as during the session to account for possible changes of the impedance of the electrode-scalp contact. The system 100 includes a self-adjusting EEG wearable 102 that can change shape and size and can autonomously establish and maintain contact between the electrodes 104 and the scalp during setup and use.

In one embodiment, as shown in FIG. 4, the cap 110 of the wearable 102 integrates linear actuators 112 between frontal, rear and side electrode segments. The linear actuators 112, as shown in FIG. 5, adjust a shape of the wearable 102 including cranium diameter and cranium height. The wearable 102 may also integrate the linear actuators 114, as shown in FIG. 6, at each of the electrode 104 that can advance the electrode 104 closer to the scalp to improve contact. The linear actuators 114 might accommodate pressure sensors to continuously monitor pressure between electrode and scalp to maximize user's comfort. The linear actuators 114 might also accommodate linear displacement sensors to continuously monitor linear displacement of the electrode relative to the frame and other electrodes for precise capture of electrode locations. The linear actuators 112, 114 may be activated via, for example, stepper motors 120. The processing device 106 may precisely control a linear displacement such that a position of the electrodes 104 is known at all times. As shown in FIG. 7, the stepper motors 120 may also open and close electrode protrusions 116 to drive the electrodes 104 through the hair and, in another embodiment, may include a vibration motor 118 to drive the electrodes 104 through the hair using mechanical vibrations, as shown in FIG. 8 and to guide the user by a tactile feedback to an electrode that might need manual adjustment by hand. The wearable 102 may also integrate a graphical interface such as, for example, a smartphone GUI 124 that monitors adjustment procedure and communicates with the user, as shown in FIG. 9.

As shown in FIG. 10, according to an exemplary method 200 utilizing the system 100, the user may be guided to set up the wearable 102 via the following autonomous procedure. In a step 202, prior to use, the cap 110 of the wearable 102 is adjusted to an initial size. In one embodiment, the initial size is a maximum size of the cap 110. In another embodiment, the maximum size of the cap is a size slightly above a size used by the user during a previous therapy session. Once the cap 110 is in the initial size, the user then places the wearable 102 over his/her head, in a step 204. In one embodiment, a chin strap of the wearable 102 may be secured under the user's chin to further secure the wearable to the user's head. A disposable adhesive reference and ground/bias electrodes may be secured to the user's ears and/or mastoids. In another embodiment, reference and ground electrodes can be embedded to the wearable 102 to provide contact with the head. In addition, one or more feedback devices 108 may be appropriately secured or attached to the user. For example, headphones may be placed over the ears or earbuds may be placed in the ears, VR headsets may be placed over the user's eyes while mechanical transducers might be placed at arbitrary location on the body for tactile feedback including vibration motors in each electrode.

In a step 206, the cap 110 of the wearable 102 may be adjusted to the user's head either automatically from the processing device 106 or via a user input. User input may include, for example, settings on a remote control, controls shown on the GUI 124 of the processing device 106, controls located on the feedback device 108, or via voice commands relayed via microphone in the processing device 106. In particular, linear actuators 112 may be extended and/or contracted, as necessary to reduce the shape and size of the cap 110 around the user's head and position the electrodes 104 in contact with the user's scalp. As described above the linear actuators 112 that change the wearable 102 shape and size may be actuated via the stepper motors 120.

In a step 208, the wearable 102 is further adjusted via driving electrodes closer to the scalp, by spreading and contracting electrode protrusions and/or by vibrating electrodes to drive electrode protrusions through the user's hair either automatically from the processing device 106 or via a user input. The linear actuators 114 that drive electrodes towards the scalp may be actuated via the stepper motors 120 at each of the electrodes 104. In a further embodiment, the stepper motors 120 also facilitate opening and/closing of the electrode protrusions 116 to further facilitate contact between the electrodes 104 and the scalp. In a further embodiment, vibration motors 118 in each electrode may be actuated from the processing device 106 to further facilitate contact between the electrodes 104 and the scalp by mechanical vibrations. The electrodes 104 may integrate pressure sensors to avoid any mechanical damage caused to the user's scalp by excessive pressure. The electrodes 104 may also integrate linear displacement sensors to precisely capture electrode location relative to the frame and other electrodes. The electrodes 104 may integrate electronics including voltage follower (unity gain amplifier) for impedance reduction, multi-color light emitting diodes (LEDs) for signaling contact quality and other electronics.

In one embodiment, vibration motors can be used to guide the user to adjust the wearable 102 so it is placed in a correct orientation on the user's head. As an example, vibration motors 118 in the pair of frontopolar electrodes are briefly activated either simultaneously or back and forth and the user is guided to improve overall the cap 110 placement by positioning frontopolar electrodes symmetrically about 1.5″ above the nasion (bridge of the nose). Next, the same procedure is repeated with occipital electrodes and the user is instructed to gain overall placement by positioning occipital electrodes symmetrically about 1.5″ above the inion (the midline bony prominence in the occipital bone) using tactile feedback from those electrodes 104. This procedure assures precise placement of the wearable over the head without necessity of precisely capturing electrode locations using optical digitizers, which is typically required in research-grade hdEEG devices.

In a step 210, when an overall placement of the headset is completed, electrode-scalp contact is assessed by measuring one or multiple features including electrode impedance between electrode and the scalp, average, root mean square (RMS) or peak-to-peak amplitude of the signal acquired from the electrode, variance of the signal, calculating power spectrum density from the acquired EEG signal, measuring power in a specific frequency range including power-line frequency (60 Hz in US, 50 Hz in Europe), measuring DC offset of the signal, measuring cross-correlation of EEG signals between neighboring electrodes, measuring phase-locking or phase coherence between signals from neighboring or distant electrodes or by measuring phase-amplitude coupling between the phase of band-pass filtered signal and amplitude of a band-pass filtered signal. The algorithm compares measured values to expected values and patterns and classifies the electrode as either with good or bad contact with the scalp.

When bad contact is detected, the algorithm may repeat steps 206 and/or 208, as necessary. In particular, contact may be improved by activating the linear actuator 114 in the electrode to drive the electrode back and forth to improve the contact, activating stepper motors to also open and close electrode protrusions to drive them through the hair, activate the vibration motor in the electrode 104 to drive the electrode protrusions 116 through the hair. The method 200 may then repeat step 210 to briefly activate vibration motors 118 in the electrodes 104 to guide the user to manually adjust the contact by pressing the electrode housing towards the scalp while turning it sideways or alternatively, by parting hair away from the electrode 104.

If system is unable to improve contact between electrode and scalp either using automatic or manual corrective approaches, electrode can be excluded from further processing.

Electrode adjustments might be performed during wearable setup, for example before a neurofeedback session, as well as in arbitrary times during use, as needed. In one embodiment, if user's discomfort is detected by user's direct input through voice commands, using, for example, the GUI 124, remote control or automatically by assessment of user's brain activity, the wearable 102 might adjust electrode locations for example by reducing pressure of electrodes against the scalp using the linear actuators 114 or by briefly activating vibration motors embedded in electrodes to massage points of contact between electrode and scalp that might become sensitive after prolonged used of the wearable. In another embodiment, vibration motors embedded in each electrode can be used to deliver head massage by activating arbitrary patterns of vibration motor activation, for example in wave patterns proceeding from front to the back of the head.

Although the system 100 is shown and described as including the linear actuators 112, 114, as shown in FIGS. 11A-D, a neurofeedback system 300 according to another exemplary embodiment may be substantially similar to the system 100, comprising a wearable 302 including a plurality of EEG electrodes 304 (e.g., 40-channel dry EEG electrodes, gelled electrodes or saline-infused electrodes) on a cap 310, the electrodes 304 configured to transmit signals to a processing device 306. The cap 310, however, may be configured to accommodate a variety of head sizes and, in one embodiment, may be configured to be stretched over a head of a user. In this embodiment, the cap 310 may be manufactured in a variety of sizes to accommodate all head sizes. In one particular embodiment, the cap 310 may be manufactured in three different sizes. The wearable 302 may be adjusted via a semi-autonomous electrode adjustment procedure in which a user manually adjusts the headset orientation and electrode-scalp contacts using tactile feedback and audio guidance through headphones.

In this embodiment, each of the electrodes 304, integrates a vibration motor in each electrode that can be individually activated for an arbitrary period of time using remote command. The motor has limited supply current to limit the possibility of damaging the scalp as well as pressure sensors for detecting the amount of electrode pressure against the scalp. The subject may place the headset over her head, secure the chin strap and insert headphones for guidance. Next, the subject is guided through the adjustment procedure, where a pair of frontopolar electrodes will be briefly (˜500 ms) activated and the subject will be guided to improve cap placement by positioning frontopolar electrodes symmetrically about 1.5″ above the nasion (bridge of the nose). Next, we will repeat the same procedure with occipital (O1, O2) electrodes and instruct the subject to place the electrodes symmetrically about 1.5″ above the inion (the midline bony prominence in the occipital bone) based on tactile feedback from those electrodes. The 1.5″ distance corresponds to 10% of the nasion-inion distance of a medium-sized (most common) cranium. EEG sensor locations may be additionally acquired using, for example, an inexpensive, 3D camera-based digitizer Structure Sensor Mark II by Occipital, which allows precise capture of all sensor locations in less than 5 minutes.

In one exemplary embodiment, electrode-scalp contact can be improved by guiding the subject to manually adjust the contact by pressing the sensor towards the scalp while turning it sideways or alternatively, by parting hair away from the electrode. After adjusting headset orientation on the subject's head, we measure the impedances of all electrodes and then guide the subject to sequentially adjust the electrodes with unsatisfactory impedances by activating the vibration motors and/or visual display of the electrodes that need adjustment one at a time, so the subject can adjust them.

The present application is directed to a precision psychiatry application to assess biologically-based, anatomically-localized functional patterns of brain network activity such as the default mode network (DMN) that has been associated with brain disorders including depression, anxiety, addiction disorders, obsessional disorders, bipolar disorder, attention-deficit/hyperactivity disorder, post-traumatic stress disorder, mild traumatic brain injury, schizophrenia and autism. The system is going to be used by therapists, mental wellness studios and at homes for both precise assessment of functional brain networks, for closed-loop non-pharmacological interventions such as neurofeedback as well as for performance enhancement.

According to one exemplary embodiment, the system of the present disclosure is in the form of a cap with integrated EEG sensors, EEG electronics (e.g., amplifiers, analog-to-digital converters and microcontroller) and slot for smartphone, which operates software for EEG processing, brain imaging (source localization), management of treatment protocols, communication with cloud-based storage and processing servers and output to variety of devices including video screens, audio devices (headphones), VR headsets, tactile transducers, intra-venous (IV) drips, pill or liquid delivery systems or inhalers for closed-loop drug delivery.

Exemplary embodiments use linear actuators to change headset shape including circumference and height, linear actuators that can drive electrodes towards the scalp and linear actuators that can open and close dry electrode protrusions to better penetrate hair and create contact with skin. Furthermore, it uses vibration motors embedded in electrodes than can help adjustment either automatically, where the algorithm briefly activates vibration motors to help the electrode tips penetrate through hair to improve contact between electrode and scalp or manually, by guiding the user to a specific electrode that needs adjustment by pressing the electrode against the scalp, twisting it and/or parting the hair from the electrode. Tactile feedback from vibration motors can also help user identify if the electrode cap is placed symmetrically, by activating vibration motors on symmetrical electrodes on the left and right side of the head and asking the subject to adjust placement so the tactile signals are perceived as symmetrical relative to the head midline. Finally, vibration motors can be used for tactile feedback during a session, for example by signaling wanted or unwanted brain states or creating point of focus or delivering a massage.

The system takes advantage of inexpensive smartphone technology to perform source localization, which significantly reduces cost of the device and enables therapist- and consumer-oriented applications.

Functional patterns of brain activity detected as frequency-specific spatial coactivation patterns in time (4-D) using methods including independent component analysis (ICA) provide independent validation of signal fidelity. If no pattern is present, EEG signal quality is not sufficient to decode a given functional brain network. This feature is comparable to detecting a pattern of power spectra using EEG signal, although EEG spectra are disembodied from brain regions which generate the underlying EEG activity and therefore this feature establishes the presence of the specific anatomically localized functional brain network from which the assessment or the neurofeedback signal is derived.

Activity within functional brain networks can be used as functional biomarkers for variety of biotypes including rumination, anhedonia, anxious avoidance, negative bias, threat dysregulation, context insensitivity, inattention and cognitive dyscontrol. As an example, rumination can be defined as increased connectivity (temporal coactivity) between nodes of default mode network observed at rest. Pattern of activity and synchronization between nodes of functional brain networks provide multidimensional biomarker which can be used for disorder classification, monitoring treatment efficacy and guiding recommendations for treatment.

Obtained activity in functional brain networks can be used for source-based neurofeedback. The system acquires EEG data and in real-time performs source localization by converting the voltage time series obtained from EEG sensors into spatially-distributed potentials in the cerebral volume using source localization methods such as the Minimum Norm Imaging method with the Dynamic Statistical Parametric Mapping (dSPM) measure or the Linearly Constrained Minimum Variance (LCMV) beamformers method. Approximately 2 minutes of baseline data obtained during rest are submitted to the temporal or spatial Independent Component Analysis (tICA, sICA) algorithm, which identifies independent spatial activation patterns that can be matched using spatial correlation to refence spatial activation patterns of functional brain networks that have been identified previously using hdEEG or fMRI. Additionally, a temporal expression of frequency-specific spatial activation patterns can be used to identify functional brain networks. Source localized activity in the form of a frequency- and location-specific signal power is classified into previously identified functional patterns of brain activity, as shown in FIG. 12, including default mode network, salience network, positive and negative affect networks, cognitive control network and attention network using specific coactivity patterns. As an example, default mode network (DMN) is characterized by coactivation of multiple anatomically defined nodes including posterior cingulate cortex (PCC), anterior medial prefrontal cortex (aMPFC) and angular gyms (AG). Spatial symmetry across brain midline axis in brain structures such angular gyms (AG) that are expressed in both left and right hemisphere can be used as an independent validation of wearable placement.

Spatial activation patterns of functional brain networks can be elicited using tasks that the user might be asked to engaged in. For example, user might be asked to rest with eyes closed or to engage into autobiographic recollection of past experiences in order to activate DMN, which pattern of activity can then be detected by the wearable.

Patterns of activity can be used as functional biomarkers for variety of biotypes including rumination, anhedonia, anxious avoidance, negative bias, threat dysregulation, context insensitivity, inattention and cognitive dyscontrol.

Averaged activity from one or multiple regions of interest that correspond to a spatial activation pattern of one or multiple functional brain networks and/or synchronization (amplitude, phase, phase-amplitude) between two or multiple nodes of functional brain networks or arbitrary independent components derived from baseline data can be used for deriving a neurofeedback signal that can be delivered to the user directly by means of audible information using headphones, as a visual information using a video screen or VR headset or through tactile feedback using tactile transducers, allowing source-based neurofeedback training, where user trains to volitionally control the neurofeedback signal and with it also activity in a specific brain region from which the neurofeedback signal was derived.

In another scenario, a signal derived from source-localized brain activity can be used to control pharmacological intervention including intra-venous (IV) drips, pill and liquid management systems or inhalers for closed-loop drug delivery. Furthermore, the signal derived from source-localized brain activity can be used to control spatial location and intensity of various types of neurostimulators including transcranial magnetic stimulation (TMS), transcranial direct current stimulation (tDCS), transcranial alternating current stimulation (tACS), electroconvulsive therapy (ECT), vagus nerve stimulation (VNS), deep brain stimulation (DBS) and other methods.

In another scenario, a signal derived from source-localized brain activity can be used to control audio-visual, cinematic or virtual reality (VR) or gaming experience in an effort to change activity of a functional brain network from which the signal is derived from unwanted to wanted activity.

The presently disclosed technology solves two major problems in treatment of brain disorders including depression and anxiety. First, it provides precise assessment using biomarkers derived from functional brain networks. Therapeutic interventions such as pharmacological drugs or talk therapy are currently administered blindly based on subjective experience of the user or using standardized questionnaires. A therapist has no metric to assess disorder nor has she a metric to monitor the efficacy of a selected therapy over time. Second, it provides for a nonpharmacological treatment of brain disorders using source-based neurofeedback, where the user volitionally changes a signal derived from observing a specific functional brain network in order to modulate its activity. Third, it can be used as an informative signal for optimizing one's experience by modifying what the user sees, hears, touches and otherwise senses, for optimizing the user's living environment and for modulation of the user's mood and feelings by means of pharmacological and non-pharmacological interventions.

The presently disclosed technology is designed to overcome a fundamental limitation of traditional EEG-based biomarkers obtained using consumer-oriented EEG devices that are typically equipped with 1-5 EEG sensors and which produce biomarkers that are based on frequency-specific power estimates (such as EEG alpha, theta, beta or gamma bands) that cannot distinguish the sources of the recorded EEG activity, which can originate in spatially separated and functionally distinct brain regions, or even from outside of the brain as is the case of muscle activity. By transforming effectively disembodied scalp EEG voltage recordings into real-time estimates of the anatomically-localized neural activity from functional brain networks, it becomes possible to routinely assess the activity that might be exaggerated in many brain disorders. To this day, high density EEG systems that can perform source localization are only used in research laboratories and rarely in neurological departments in hospitals. The real-time source localization capability is not routinely utilized in any therapeutic application. An EEG headset that can perform real-time source localization of EEG signals and that can be setup without the help of trained personnel does not exist.

Applications include neuropsychiatric assessment based on activity in functional brain networks and source-based neurofeedback. The assessment is used for neuropsychiatric diagnostics but also tracking therapeutic progress in a variety of therapies including pharmacological treatment, talk therapy, group therapy, neurostimulation, and neurofeedback.

In the neurofeedback application, an activity in a specific functional brain network influences audio or video information or controls tactile or other sensory feedback delivered to a patient in order to train the patient to avoid or engage in a particular brain state. Source-based neurofeedback might be used for nonpharmacological treatment of a variety of brain disorders including depression, anxiety, addiction disorders, obsessional disorders, bipolar disorder, attention-deficit/hyperactivity disorder, post-traumatic stress disorder, mild traumatic brain injury, schizophrenia and autism.

Other applications may also include enhanced talk therapy where a human or an Al-based therapist uses real-time information obtained from functional brain networks to support the therapy. As an example, a therapist might use the technology to continuously monitor the negative affect network in a patient that is being treated for post-traumatic stress disorder (PTSD). High activity in the negative affect network would notify the therapist about an experience potentially associated with trauma.

Other further applications might include sex therapy for treatment of sexual dysfunction including anorgasmia in women or erectile dysfunction in men, where the system can train the user to reach a particular brain state to promote sexual function.

Other yet further applications might include cognitive enhancement of individuals including improvement of focus, resilience, cognitive control and flexibility, learning and information processing, selective, sustained, divided and simultaneous attention, pattern recognition, category formation, response inhibition, short- and long-term memory, visual and auditory processing, planning, communication, logical reasoning, self-awareness, consciousness, induction of “flow states” and social skills. As an example, user equipped with device can train their mind to stay in a given brain state such as sustained attention to an object in the physical or virtual room. When attention is weakened, user can be notified to resume attention.

Other further applications might include enhancement of group cognitive functioning including improvement of group cohesion in teams and classes of students, improvement in motivation, communication, cognitive load distribution and processing, group cognitive flexibility, hierarchical order execution and distributed problem solving. Such technology can be applied in schools, corporations, first responders and in the battlefield. As an example, team of drone operators can be equipped with the device to monitor optimal performance such as attention to environment and commands. If lower performance is detected in an operator, team degradation can be prevented by on-the-fly replacement of the operator. In a different scenario, a teacher can receive real-time feedback from a class of students equipped with devices which can signal the group's attention to the subject that is being taught.

Other further applications might include enhancement of human-machine interaction to maximize transfer of information between machine and human, creating optimal information flow from machine to human to maximize a human's information processing and understanding and minimize response times. As an example, communication can be preferentially delivered only to moments of heightened attention to maximize likelihood of understanding.

Other further applications might include various types of group sessions including yoga and meditation classes, prayers, group therapy sessions and circles, where a leader of the group can sense the overall group experience and can identify those individuals that need special attention. In one scenario, signals derived from functional brain networks of one or multiple users can be converted into a perceivable signal using audio, video, tactile, or other sensory inputs delivered to the group or to individuals as a part of therapy.

Other further applications might include improvement of gaming skills for individuals and teams.

1-23. (canceled)

24. A system for precision psychiatry, comprising:
a wearable device including a cap and a plurality of EEG electrodes attached thereto, the cap configured to be placed over a head of a user and including a plurality of first linear actuators, each of the first linear actuators configured to expand and contract to increase and decrease a size of the cap, respectively, the EEG electrodes connected to the cap via a plurality of second linear actuators, each of the second linear actuators configured to expand and contract to move at least one of the EEG electrodes toward and away from, respectively, a scalp of the user when the cap is placed over the head of the user while a 3-D location of the EEG electrodes is known at all times;
a processing device configured to be connected to the EEG electrodes of the wearable device to know the 3-D location of the EEG electrodes and to receive brain activity signals therefrom, the processing device generating a neurofeedback signal based on a detected aberrant brain activity; and
a feedback device providing the neurofeedback signal to the user.

25. The system of claim 24, wherein the processing device autonomously controls an adjustment of the wearable device such that the EEG electrodes contact the scalp of the user.

26. The system of claim 24, wherein the processing device includes EEG electronics and a smartphone.

27. The system of claim 26, wherein the processing device includes a graphical user interface displaying and monitoring an adjustment of the wearable device.

28. The system of claim 24, wherein the feedback device includes one of a headphone, an carbud, a video screen, a VR headset and a tactile transducer.

29. The system of claim 24, wherein the neurofeedback signal includes one of audible information, visual information, tactile information and pharmacological intervention.

30. The system of claim 24, wherein at least one of the second linear actuators activates to control a movement of electrode protrusions relative to one another to further adjust a contact between the EEG electrodes and the scalp.

31. The system of claim 24, wherein each of the EEG electrodes include a vibration motor for vibrating the EEG electrodes to further adjust a contact between the EEG electrodes and the scalp.

32. The system of claim 24, wherein the cap is 3D printed.

33. The system of claim 24, further comprising EEG electronics including a flexible printed circuit board (PCB).

34. A high-density EEG wearable device for precision psychiatry, comprising:
a cap configured to be placed over a head of a user, the cap including a plurality of first linear actuators, each of the first linear actuators configured to expand and contract to increase and decrease a size of the cap, respectively; and
a plurality of EEG electrodes attached to the cap, the EEG electrodes connected to one another via the first linear actuators and to the cap via a plurality of second linear actuators, each of the second linear actuators configured to expand and contract to move the EEG electrode toward and away from, respectively, a scalp of the user when the cap is placed over the head of the user.

35. The device of claim 34, wherein the first and second linear actuators are activated via a stepper motor.

36. The device of claim 35, wherein activation of the stepper motor actuates each of the second linear actuators to move a plurality of protrusions of each of the EEG electrodes.

37. The device of claim 35, wherein each of the EEG electrodes includes a vibration motor configured to vibrate the EEG electrodes.

38. The device of claim 35, wherein the EEG electrodes are configured to transmit brain activity signals to a processing device via one of a wired and a wireless connection.

39. A method for autonomously adjusting a high-density EEG wearable device, comprising:
adjusting a cap of a wearable device, situated on a head of a user, from an initial configuration to a further configuration,
wherein during the further configuration a plurality of EEG electrodes attached to the cap contacting a scalp of the user, and
wherein the adjusting step includes at least one of the following substeps:
adjusting a size of the cap via one of an expansion and contraction of at least one of first linear actuators connecting the EEG electrodes to one another, and
adjusting a position of at least one of the EEG electrodes relative to the scalp via one of an expansion and a contraction of at least one of second linear actuators connecting the EEG electrodes to the cap.

40. The method of claim 39, wherein the cap is set to the initial configuration prior to placing the cap over the head of the user.

41. The method of claim 39, wherein adjusting the position of at least one of the EEG electrodes relative to the scalp includes activating the second linear actuators so that electrode protrusions of each of the EEG electrodes moves relative to one another.

42. The method of claim 39, further comprising vibrating one or more of the EEG electrodes to further adjust the position of at least one of the EEG electrodes relative to the scalp.

43. The method of claim 42, wherein a vibration of at least one of the EEG electrodes guides the user to move at least one of the EEG electrodes to an optimal position along the scalp.

44. The method of claim 39, further comprising assessing a contact between the EEG electrodes and the scalp.

45. The method of claim 44, wherein assessing the contact between the EEG electrodes and the scalp includes measuring one or more multiple features including electrode impedance between the EEG electrodes and the scalp, average, root mean square, peak-to-peak amplitude of a signal acquired from at least one of the EEG electrodes, variance of the signal, calculating power spectrum density from the acquired signal, measuring power in specific frequency range including power-line frequency, and measuring DC offset of the signal or measuring cross-correlation of signals between neighboring electrodes, measuring phase-locking or phase coherence between signals from neighboring or distant electrodes or by measuring phase-amplitude coupling between phase of hand-pass filtered low frequency signal and amplitude of band-pass filtered high frequency signal.

46. The method of claim 45, wherein assessing the contact between the EEG electrodes and the scalp includes comparing measured values to expected values.

47. The method of claim 45, wherein assessment of a contact quality between the EEG electrodes and the scalp is performed during use and adjusted automatically or semi-automatically.
